(19) United States
(12) Patent Application Publication
(10) Pub. No.: US 2025/0032094 A1
(43) Pub. Date: Jan. 30, 2025

(54) INFERENCE APPARATUS, ULTRASOUND DIAGNOSTIC APPARATUS, TRAINING APPARATUS, INFERENCE METHOD, DISPLAY METHOD, AND PROGRAM

(71) Applicant: KONICA MINOLTA, INC., Tokyo (JP)

(72) Inventor: Mutsuhiro AKAHANE, Tokyo (JP)

(73) Assignee: KONICA MINOLTA, INC., Tokyo (JP)

(21) Appl. No.: 18/776,043

(22) Filed: Jul. 17, 2024

(30) Foreign Application Priority Data
Jul. 24, 2023 (JP) 2023-119918

(51) Int. Cl. A61B 8/08; A61B 8/00; A61B 8/06; G06T 7/00; G06T 7/70

(52) U.S. Cl. CPC A61B 8/5207; A61B 8/06; A61B 8/085; A61B 8/463; G06T 7/0012; G06T 7/70; G06T 2207/10132; G06T 2207/20081; G06T 2207/20084; G06T 2207/30012; G06T 2207/30104

(57) ABSTRACT

Provided is an inference apparatus according to the present disclosure including: a hardware processor that: acquires ultrasound image data on an ultrasound image generated based on a reflected wave of ultrasound transmitted to a subject; and inputs the ultrasound image data, as data to be inferred, to a trained model and performs inference on a nerve root in the ultrasound image based on output data outputted from the trained model.

### CROSS REFERENCE TO RELATED APPLICATIONS

The entire disclosure of Japanese Patent Application No. 2023-119918, filed on Jul. 24, 2023, is incorporated herein by reference in its entirety.

### BACKGROUND

### Technological Field

The present disclosure relates to an inference apparatus, an ultrasound diagnostic apparatus, a training apparatus, an inference method, a display method, and a program.

### Description of the Related Art

With the recent development of deep learning technology, machine learning models have been utilized for various purposes. For example, Japanese Unexamined Patent Application Publication (Translation of PCT Application) No. 2020-519369 and Japanese Patent Application Laid-Open No. 2021-164573 have proposed the utilization of a machine learning model for image diagnosis of ultrasound image data in the medical field.

### SUMMARY

For a patient suffering from lower back pain, a lumbar nerve root block is performed. Generally, a nerve root block is an injection of an anesthetic liquid into a lumbar nerve root. Generally, a nerve root block is executed by puncture under X-ray fluoroscopy, but a nerve root itself is not imaged. For this reason, the doctor determines that a puncture needle has reached the vicinity of a nerve root, by bringing the puncture needle into contact with the nerve root and observing a pain response of the patient.

However, since such a method imposes a heavy burden on the patient, improvement is desired.

In recent years, a technique has been developed in which when a nerve root block for a cervical nerve root is performed, puncture is performed while the position of the nerve root is confirmed in real time by using an ultrasound diagnostic apparatus.

In a case where a lumbar nerve root is rendered using an ultrasound diagnostic apparatus, the fifth lumbar nerve root (also referred to as the L5 nerve root), for which puncture is particularly highly frequently performed, is located in a deep position from the body surface and is surrounded by bones, and therefore, there is a problem in that it is difficult to render the nerve root as compared with a cervical nerve root. Further, in a case where a lumbar nerve root is observed from the back, the nerve root is observed through a muscle such as the multifidus muscle or the erector spinae muscle. For a patient whose muscles have turned into fat due to aging, ultrasound is scattered due to the fat, which makes it more difficult to render a lumbar nerve root.

In consideration of such circumstances, an object of the present disclosure is to provide an inference apparatus capable of performing accurate inference on a nerve root by using a trained model, and an ultrasound diagnostic apparatus, a training apparatus, an inference method, a display method, and a program which are related to the inference apparatus.

To achieve at least one of the abovementioned objects, according to an aspect of the present invention, an inference apparatus according to an aspect of the present disclosure includes a hardware processor that: acquires ultrasound image data on an ultrasound image generated based on a reflected wave of ultrasound transmitted to a subject; and inputs the ultrasound image data, as data to be inferred, to a trained model and performs inference on a nerve root in the ultrasound image based on output data outputted from the trained model.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a schematic diagram illustrating training processing and inference processing in an ultrasound diagnostic system;

FIG. 2 is a schematic diagram illustrating Variation 1 of the training processing and the inference processing according to the present disclosure;

FIG. 3 is a schematic diagram illustrating Variation 2 of the training processing and the inference processing according to the present disclosure;

FIG. 4 is a diagram illustrating an exemplary appearance of the ultrasound diagnostic apparatus according to an embodiment of the present disclosure;

FIG. 5 is a block diagram illustrating a configuration example of the ultrasound diagnostic apparatus according to the embodiment of the present disclosure;

FIG. 6 is a block diagram illustrating a configuration example of a training apparatus according to the embodiment of the present disclosure;

FIG. 7 is a block diagram illustrating a hardware configuration example of an inference apparatus and the training apparatus according to the embodiment of the present disclosure;

FIG. 8 is a flowchart provided for describing an operation example of the training apparatus in a training phase;

FIG. 9 is a diagram illustrating an exemplary ultrasound image of the lumbar region;

FIGS. 10A and 10B are diagrams illustrating other exemplary ultrasound images of the lumbar region;

FIGS. 11A and 11B are diagrams illustrating other exemplary ultrasound images of the lumbar region;

FIGS. 12A and 12B are diagrams illustrating other exemplary ultrasound images of the lumbar region;

FIG. 13 is a diagram illustrating an exemplary ultrasound image of the lumbar region including blood flow information;

FIG. 14 is a flowchart provided for describing an operation example of the ultrasound diagnostic apparatus in an inference phase;

FIG. 15 is a diagram illustrating an exemplary display image; and

FIG. 16 is a diagram illustrating other exemplary display images.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, an embodiment of the present disclosure will be described in detail with reference to the accompanying drawings.

### Outline of the Present Disclosure

Hereinafter, an ultrasound diagnostic system is disclosed which includes: a training apparatus that trains a machine learning model (a model to be trained) for performing inference on a nerve root in an ultrasound image; an inference apparatus that performs inference on a nerve root by utilizing a trained model; and an ultrasound diagnostic apparatus that generates an ultrasound image to be inferred and displays the ultrasound image based on an inference result.

The trained model trained by the training apparatus is trained so as to output, by using ultrasound image data on an ultrasound image including a bone existing around a nerve root as training data, output data in a case where the ultrasound image data is inputted.

The ultrasound image apparatus includes an inference apparatus that holds a trained model for which the training by the training apparatus has been completed. The ultrasound image apparatus transmits ultrasound to a subject, generates an ultrasound image based on reflected ultrasound, and passes ultrasound image data on the ultrasound image to the inference apparatus. The inference apparatus inputs the ultrasound image data to the trained model and performs inference on a nerve root based on output data that has been outputted. Specifically, the inference apparatus performs inference on the position of a nerve root in an ultrasound image.

The ultrasound image apparatus displays an ultrasound image based on an inference result outputted by the inference apparatus. Specifically, the ultrasound image apparatus displays, together with an ultrasound image, the position of a nerve root based on an inference result.

Accordingly, a user (a doctor or the like) who uses the ultrasound diagnostic system can perform a nerve root block while confirming the position of a nerve root at any time based on an ultrasound image. In particular, when the training apparatus trains a model to be trained, by using, as training data, an ultrasound image including at least one bone of the fifth lumbar vertebra, the fourth lumbar vertebra or the sacrum, each of which exists around a lumbar nerve root, it is possible to perform a lumbar nerve root block with a relatively small burden on the subject.

### System Configuration

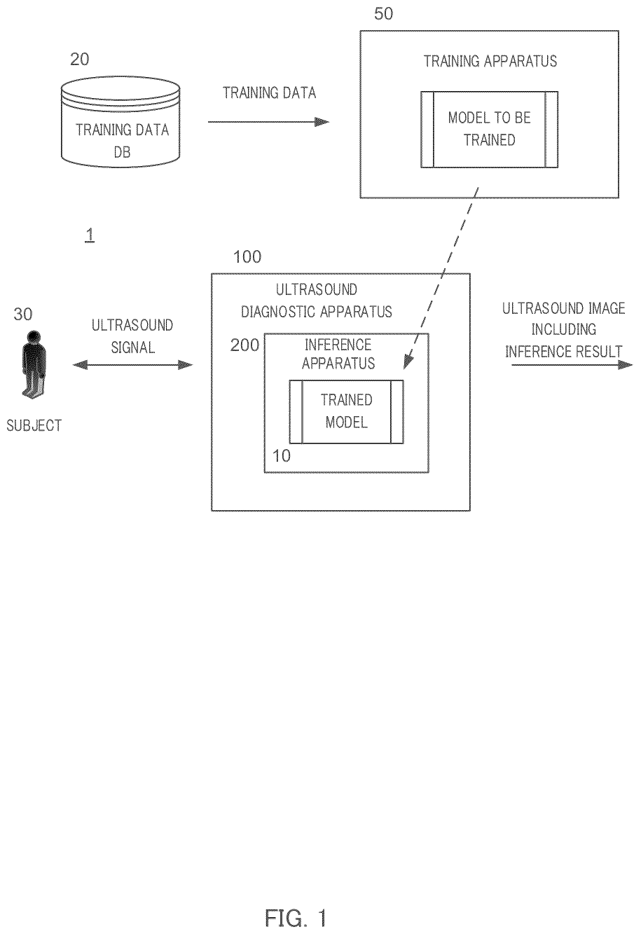

First, a system configuration of ultrasound diagnostic system 1 according to an embodiment of the present disclosure will be described with reference to FIG. 1. FIG. 1 is a schematic diagram illustrating training processing and inference processing in ultrasound diagnostic system 1.

As illustrated in FIG. 1, ultrasound diagnostic system 1 includes training apparatus 50 and ultrasound diagnostic apparatus 100. Ultrasound diagnostic apparatus 100 includes inference apparatus 200.

Training apparatus 50 stores a machine learning model to be trained, and uses training data stored in training data database (DB) 20 to train the machine learning model to be trained. The machine learning model to be trained may be realized as a machine learning model of an arbitrary suitable type, such as a neural network, for example. For example, in a case where the machine learning model to be trained is realized by a neural network, training apparatus 50 may execute supervised learning by using training data acquired from training data DB 20 and update a parameter(s) of the machine learning model according to an arbitrary training algorithm known in the art, such as a backpropagation method.

When the training of the machine learning model is completed, the trained machine learning model (hereinafter referred to as trained model 10) is stored in a storage unit of inference apparatus 200 included in ultrasound diagnostic apparatus 100, or the like.

Ultrasound diagnostic apparatus 100 generates ultrasound image data by transmitting and receiving an ultrasound signal to and from subject 30 via an ultrasound probe.

Inference apparatus 200 inputs ultrasound image data to trained model 10 and performs inference on a nerve root in an ultrasound image, for example, inference on the position of the nerve root within the image, or the like, based on output data that has been outputted.

Ultrasound diagnostic apparatus 100 displays an ultrasound image and performs displaying on a nerve root included in the ultrasound image based on an inference result of inference apparatus 200.

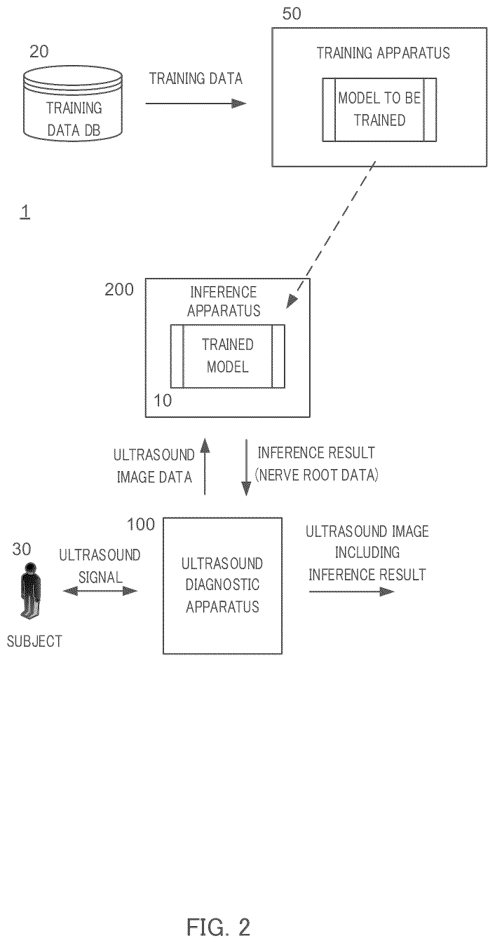

Although the system configuration according to an embodiment of the present disclosure has been described with reference to FIG. 1, the system configuration according to the present disclosure is not limited thereto. FIG. 2 is a schematic diagram illustrating Variation 1 of the training processing and the inference processing according to the present disclosure. In the example illustrated in FIG. 2, ultrasound diagnostic apparatus 100 and inference apparatus 200 are independent of each other. In this case, ultrasound diagnostic apparatus 100 outputs generated ultrasound image data to inference apparatus 200, and inference apparatus 200 returns, to ultrasound diagnostic apparatus 100, output data that is a result of inference performed by using trained model 10. Thus, based on the inference result of inference apparatus 200, ultrasound diagnostic apparatus 100 can display an ultrasound image and performs displaying on a nerve root included in the ultrasound image.

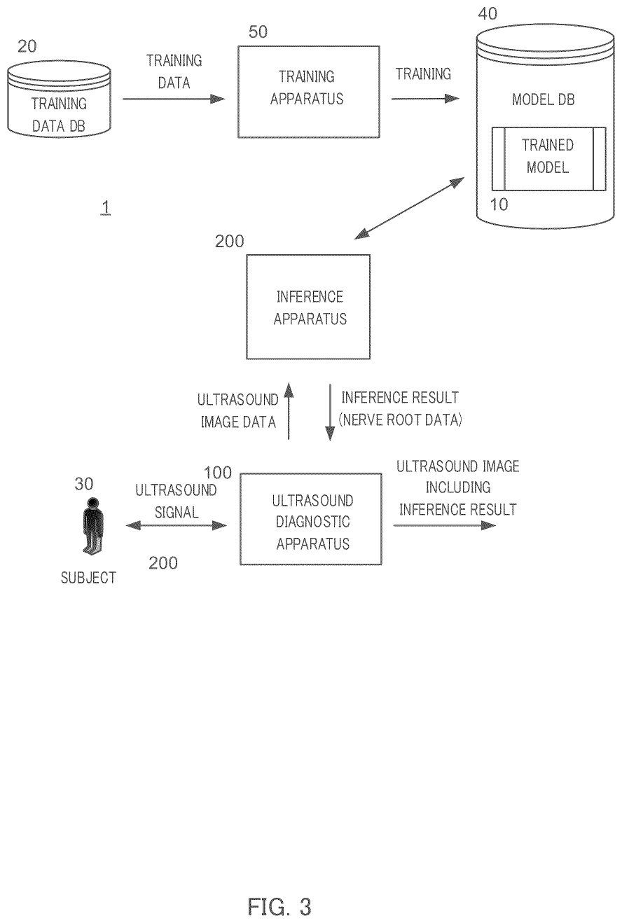

FIG. 3 is a schematic diagram illustrating Variation 2 of the training processing and the inference processing according to the present disclosure. In the example illustrated in FIG. 3, training apparatus 50 may train a machine learning model to be trained that is stored in model database (DB) 40, and trained model 10 for which the training has been completed may be stored in model DB 40. Inference apparatus 200 accesses model DB 40 and acquires output data by utilizing trained model 10. According to Variation 2 as such, even in a case where inference apparatus 200 does not include a storage resource for storing trained model 10, an effect equivalent to that of the embodiment illustrated in FIG. 1 can be achieved by utilizing trained model 10 stored in model DB 40.

Further, although illustration is omitted, the present disclosure may include a form in which an ultrasound diagnostic apparatus that generates an ultrasound image and an image display apparatus that displays an ultrasound image based on an inference result of inference apparatus 200 are separately provided.

### Configuration of Ultrasonic Diagnostic Apparatus 100

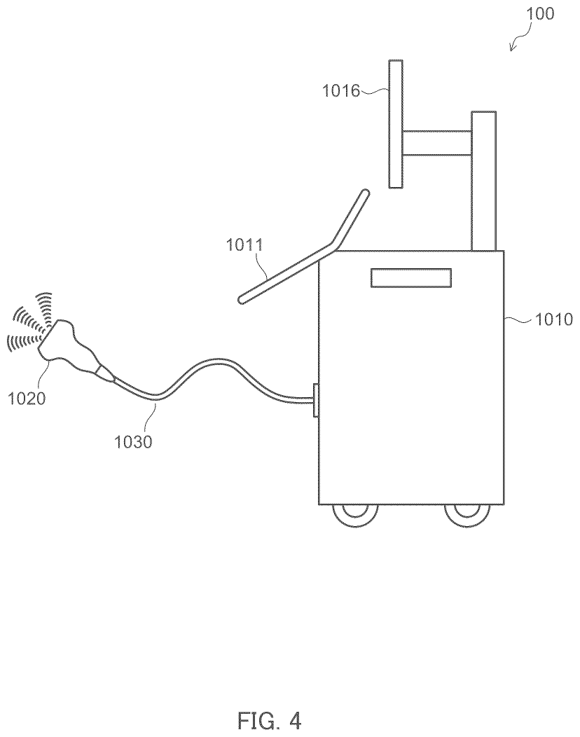

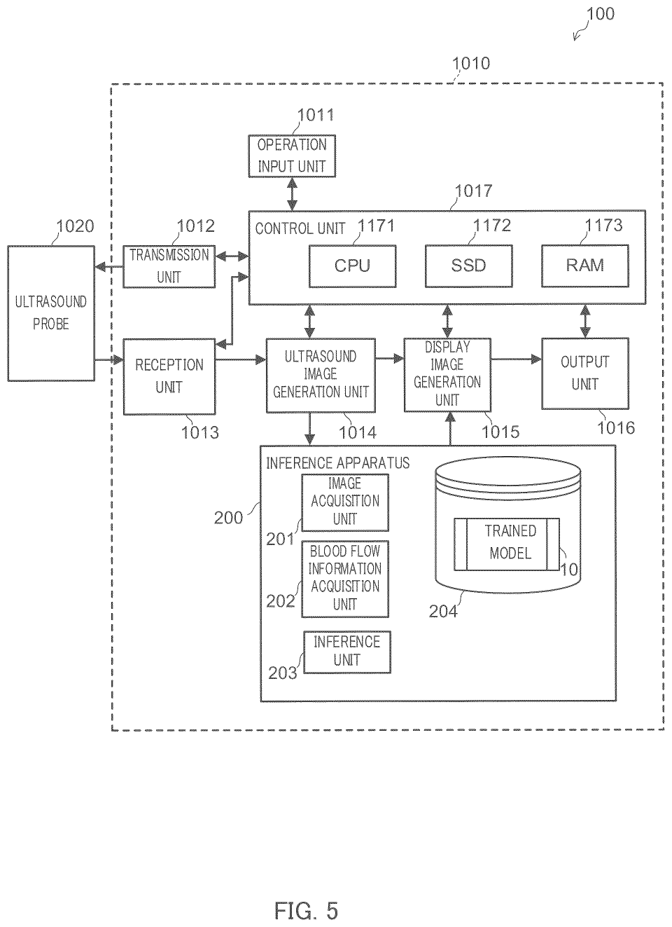

FIG. 4 is a diagram illustrating an exemplary appearance of ultrasound diagnostic apparatus 100 according to the embodiment of the present disclosure. FIG. 5 is a block diagram illustrating a configuration example of ultrasound diagnostic apparatus 100 according to the embodiment of the present disclosure.

Ultrasound diagnostic apparatus 100 visualizes, as an ultrasound image, at least one of a shape and/or a dynamic state within subject 30. Ultrasound diagnostic apparatus 100 is used, for example, in applications to capture an ultrasound image (i.e., a tomographic image) of a region to be detected, in particular the lumbar region, and to perform a nerve root block for a nerve root existing in the region to be detected, in particular a lumbar nerve root.

As illustrated in FIG. 4, ultrasound diagnostic apparatus 100 includes ultrasound diagnostic apparatus main body 1010 and ultrasound probe 1020. Ultrasound diagnostic apparatus main body 1010 and ultrasound probe 1020 are connected to each other via cable 1030.

Ultrasound probe 1020 functions as an acoustic sensor that transmits an ultrasound beam to subject 30 (e.g., a patient), receives a reflected wave (ultrasound echo) of the transmitted ultrasound beam, which is reflected inside the body of subject 30, and converts the reflected wave into an electric signal.

The user operates ultrasound diagnostic apparatus 100 by bringing an ultrasound beam transmission/reception surface of ultrasound probe 1020 into contact with the body surface of a region to be detected of subject 30. Note that, as ultrasound probe 1020, an arbitrary ultrasound probe such as a convex probe, a linear probe, a sector probe, or a three-dimensional probe can be applied.

Ultrasound probe 1020 is configured to include, for example, a plurality of transducers (e.g., piezoelectric elements) arranged in an array shape, and a channel switching apparatus (e.g., a multiplexer) for controlling switching to turn on/off a driving state of the plurality of transducers individually or in units of blocks.

Each transducer of ultrasound probe 1020 converts a voltage pulse generated by transmission unit 1012 of ultrasound diagnostic apparatus main body 1010 into an ultrasound beam, transmits the ultrasound beam to subject 30, receives an ultrasound echo reflected inside subject 30, converts the ultrasound echo into an electric signal, and outputs the electric signal to reception unit 1013 of ultrasound diagnostic apparatus main body 1010.

In the present embodiment, as illustrated in FIG. 5, ultrasound diagnostic apparatus main body 1010 includes operation input unit 1011, transmission unit 1012, reception unit 1013, ultrasound image generation unit 1014, display image generation unit 1015, output unit 1016, control unit 1017, and inference apparatus 200.

Transmission unit 1012, reception unit 1013, ultrasound image generation unit 1014, and display image generation unit 1015 are constituted by, for example, dedicated or general-purpose hardware (electronic circuit) supporting each processing, such as a central process unit (CPU), a digital signal processor (DSP), an application specific integrated circuit (ASIC), or a field programmable gate array (FPGA), and implement each function in cooperation with control unit 1017.

Operation input unit 1011 receives, for example, a command instructing an operation of ultrasound diagnostic apparatus 100, an input of information on subject 30, or the like. Operation input unit 1011 may include, for example, an operation panel including a plurality of input switches, a keyboard, a mouse, and/or the like. Note that, operation input unit 1011 may be constituted by a touch screen provided integrally with output unit 1016.

Transmission unit 1012 is a transmitter that transmits a voltage pulse as a driving signal to ultrasound probe 1020 according to an instruction of control unit 1017. Transmission unit 1012 may be configured to include, for example, a high-frequency pulse oscillator, a pulse setting unit, and the like. Transmission unit 1012 may adjust a voltage pulse generated by the high-frequency pulse oscillator to have a voltage amplitude, a pulse width, and a transmission timing that are set by the pulse setting unit, and transmit the voltage pulse for each channel of ultrasound probe 1020.

Transmission unit 1012 includes a pulse setting unit for each of the plurality of channels of ultrasound probe 1020, and the voltage amplitude, pulse width, and transmission timing of a voltage pulse can be set for each of the plurality of channels. For example, transmission unit 1012 may change a target depth or generate a different pulse waveform by setting an appropriate delay time for the plurality of channels.

Reception unit 1013 is a receiver that receives and processes, according to an instruction of control unit 1017, an electric signal according to an ultrasound echo generated by ultrasound probe 1020. Reception unit 1013 may include a preamplifier, an AD conversion unit, and a reception beamformer.

Reception unit 1013 amplifies a reception signal according to a weak ultrasound echo for each channel by the preamplifier, and converts the reception signal into a digital signal by the AD conversion unit. Then, reception unit 1013 uses the reception beamformer to perform delay and sum on the reception signals of the respective channels, thereby combining the reception signals of the plurality of channels into one to obtain acoustic line data.

Ultrasound image generation unit 1014 acquires a reception signal (acoustic line data) from reception unit 1013 and generates an ultrasound image (i.e., a tomographic image) of the inside of subject 30.

For example, when ultrasound probe 1020 transmits a pulsed ultrasound beam in the depth direction, ultrasound image generation unit 1014 temporally continuously accumulates the signal intensity of an ultrasound echo, which is detected thereafter, in a line memory. Then, as the ultrasound beam from ultrasound probe 1020 scans the inside of subject 30, ultrasound image generation unit 1014 sequentially accumulates the signal intensity of the ultrasound echo in each scanning position, in the line memory, and generates two-dimensional data in units of frames. Then, ultrasound image generation unit 1014 may convert the signal intensity of the two-dimensional data into a luminance value, thereby generating an ultrasound image representing a two-dimensional structure within a cross section including the transmission direction of ultrasound and the scanning direction of the ultrasound.

Note that, ultrasound image generation unit 1014 may include, for example, an envelope detection circuit that performs envelope detection on a reception signal acquired from reception unit 1013, a logarithmic compression circuit that performs logarithmic compression on the signal intensity of the reception signal detected by the envelope detection circuit, a dynamic filter that removes a noise component included in a reception signal, with a band-pass filter whose frequency characteristic is changed according to the depth, and the like.

Ultrasound image generation unit 1014 may be capable of generating, when needed, ultrasound image data in, in addition to a B mode, any other mode such as a color Doppler mode, a motion (M) mode, or a pulse Doppler mode as the image mode. The B mode is a mode for generating a tomographic image in which the intensity of a reception signal of ultrasound in a subject is represented by luminance. The color Doppler mode is a mode for converting a Doppler shift frequency generated by a blood flow of a subject into a velocity and displaying the velocity distribution as a tomographic image.

Ultrasound image data on an ultrasound image generated by ultrasound image generation unit 1014 is outputted to inference apparatus 200.

As illustrated in FIG. 5, inference apparatus 200 includes image acquisition unit 201, blood flow information acquisition unit 202, inference unit 203, and storage unit 204.

Image acquisition unit 201 acquires ultrasound image data outputted from ultrasound image generation unit 1014.

In a case where ultrasound image generation unit 1014 generates an image in the color Doppler mode, blood flow information acquisition unit 202 acquires blood flow information on a blood flow flowing in a blood vessel included in the ultrasound image.

Inference unit 203 inputs ultrasound image data, as data to be inferred, to trained model 10 stored in storage unit 204 and performs inference on a nerve root in an ultrasound image based on output data on the nerve root outputted from trained model 10. The content of the inference performed by inference unit 203 is, for example, inference on the position of a nerve root in an ultrasound image.

Output data that is a result of reference by inference unit 203 is outputted to display image generation unit 1015.

Display image generation unit 1015 generates, based on ultrasound image data generated by ultrasound image generation unit 1014 and output data acquired from inference apparatus 200, a display image including an ultrasound image and displaying on a nerve root. Then, display image generation unit 1015 transmits data of the generated display image to output unit 1016. Display image generation unit 1015 may sequentially update a display image each time a new ultrasound image is acquired from ultrasound image generation unit 1014, and cause output unit 1016 to display the display image in a moving image format.

Note that, display image generation unit 1015 may generate a display image after performing predetermined image processing, such as coordinate conversion processing or data interpolation processing, on an ultrasound image outputted from ultrasound image generation unit 1014.

Following an instruction from control unit 1017, output unit 1016 acquires data of a display image from display image generation unit 1015 and outputs the display image. For example, output unit 1016 may be constituted by a liquid crystal display, an organic EL display, a CRT display, or the like.

Control unit 1017 controls operation input unit 1011, transmission unit 1012, reception unit 1013, ultrasound image generation unit 1014, display image generation unit 1015, output unit 1016, and inference apparatus 200 according to the functions thereof, thereby performing overall control of ultrasound diagnostic apparatus 100. Note that, in the present disclosure, the control of inference apparatus 200 may not be performed by control unit 1017, and for example, inference apparatus 200 may include a control unit independent of control unit 1017.

Control unit 1017 may include central processing unit (CPU) 1171 as an arithmetic/control apparatus, solid state drive (SSD) 1172 in which a basic program and basic setting data are stored, random access memory (RAM) 1173 as a main storage apparatus, and the like. CPU 1171 reads a program corresponding to a processing content from SSD 1172, stores the program in RAM 1173, and executes the stored program, thereby centrally controlling the operation of each functional block of ultrasound diagnostic apparatus main body 1010 and inference apparatus 200.

### Configuration of Training Apparatus 50

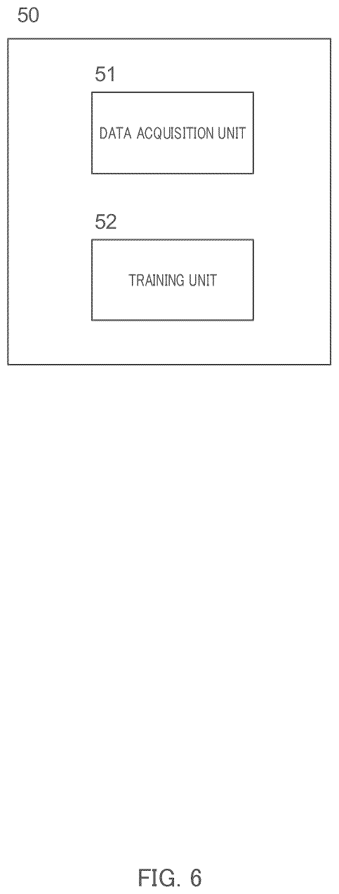

Next, a configuration of training apparatus 50 according to the embodiment of the present disclosure will be described. FIG. 6 is a block diagram illustrating a configuration example of training apparatus 50 according to the embodiment of the present disclosure.

As illustrated in FIG. 6, training apparatus 50 includes data acquisition unit 51 and training unit 52.

Data acquisition unit 51 acquires training data (learning data) that is used for training of a machine learning model to be trained. Specifically, data acquisition unit 51 acquires a pair of ultrasound image data on an ultrasound image and a ground truth label indicating the position of a nerve root in the ultrasound image.

Note that, the ultrasound image data as training data acquired by training apparatus 50 may not necessarily be the ultrasound image data generated by ultrasound diagnostic apparatus 100 described above. Training apparatus 50 may acquire, for example, ultrasound image data including a large number of ultrasound images generated in the past by another/other ultrasound diagnostic apparatus(es), and ground truth labels assigned in advance to the ultrasound images, respectively. Training apparatus 50 may acquire the training data from, for example, a company(s) or an organization(s) that provide(s) pairs of ultrasound image data and ground truth labels, or may use, for example, existent data existing on a public network, such as the Internet, as the training data.

The ground truth labels may be assigned in advance to a plurality of ultrasound images as described above, or may be assigned by the user of ultrasound diagnostic system 1 to ultrasound images, respectively, which are generated using ultrasound diagnostic apparatus 100.

Data acquisition unit 51 may perform pre-processing, such as contrast change or noise removal, on the acquired ultrasound image data for training. Furthermore, data acquisition unit 51 may execute data augmentation on the acquired training data to increase training data. For example, data acquisition unit 51 may execute data augmentation by performing image transformation processing, such as enlargement or reduction, position change, or deformation, on ultrasound image data for training.

Training unit 52 uses a pair of ultrasound image data and a ground truth label as training data to generate trained model 10 that is trained so as to output an inference result on a nerve root in response to an input of ultrasound image data.

More specifically, training unit 52 compares output data, which is outputted by a machine learning model to be trained in response to an input of ultrasound image data for training, with a ground truth label, and updates a parameter(s) of the machine learning model to be trained, based on a comparison result (error). For example, in a case where a machine learning model is realized by a convolutional neural network, training unit 52 may continue to adjust a parameter(s) of the machine learning model according to an error between an output result and ground truth data in accordance with the backpropagation method until a predetermined completion condition is satisfied.

In the present embodiment, when training unit 52 determines that the training of a machine learning model has been completed, training unit 52 provides inference apparatus 200 of ultrasound diagnostic apparatus 100 with trained model 10 (see FIG. 1).

### Hardware Configuration of Inference Apparatus 200 and Training Apparatus 50

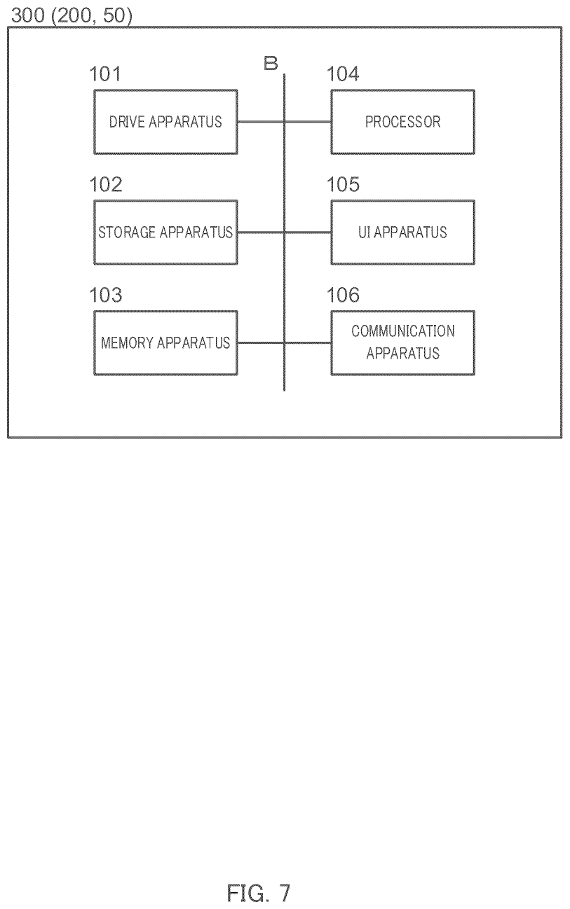

FIG. 7 is a block diagram illustrating an exemplary hardware configuration of inference apparatus 200 and training apparatus 50 according to the embodiment of the present disclosure.

Each of inference apparatus 200 and training apparatus 50 may be realized by various types of calculation apparatuses 300 such as a server apparatus, a personal computer (PC), a workstation, a smartphone, or a tablet terminal. That is, calculation apparatus 300 which may form inference apparatus 200 or training apparatus 50 includes drive apparatus 101, storage apparatus 102, memory apparatus 103, processor 104, user interface (UI) apparatus 105, and communication apparatus 106 which are interconnected via bus B.

Programs or instructions for implementing various functions and processing of inference apparatus 200 and training apparatus 50 may be stored in an attachable/detachable storage medium such as a compact disc read-only memory (CD-ROM) or a flash memory. When the storage medium is set in drive apparatus 101, a program or an instruction is installed in storage apparatus 102 or memory apparatus 103 from the storage medium via drive apparatus 101. However, the program or the instruction may not need to be installed from the storage medium, but may be downloaded from any external apparatus via a network or the like.

Storage apparatus 102 is realized by a storage apparatus such as a hard disk drive (HDD) or an SSD, and stores, together with an installed program or instruction, a file, data, or the like used for executing the program or the instruction.

Memory apparatus 103 is realized by a random access memory, a static memory, or the like, and reads, when a program or an instruction is activated, a program, an instruction, data, or the like from storage apparatus 102 and stores the read program, instruction, data, or the like. Storage apparatus 102, memory apparatus 103, and the attachable/detachable storage medium may be collectively referred to as a non-transitory storage medium.

Processor 104 may be realized by at least one central processing unit (CPU), graphics processing unit (GPU), processing circuitry, or the like, which may be constituted by at least one processor core, and executes various functions and processing of inference apparatus 200 and training apparatus 50 according to programs and instructions stored in memory apparatus 103 and data such as a parameter(s) required to execute the programs or instructions.

User interface (UI) apparatus 105 may be constituted by an input apparatus such as a keyboard, a mouse, a camera, or a microphone, an output apparatus such as a display, a speaker, a headset, or a printer, or an input/output apparatus such as a touch screen, and realizes an interface between the user and inference apparatus 200 as well as training apparatus 50. For example, the user operates a graphical user interface (GUI) displayed on a display or a touch screen with a keyboard, a mouse, or the like to operate inference apparatus 200 and training apparatus 50.

Communication apparatus 106 is realized by various communication circuits that execute wired or wireless communication processing with an external apparatus or a communication network such as the Internet, a local area network (LAN), or a cellular network.

The hardware configuration exemplified in FIG. 7 is merely an example, and inference apparatus 200 and training apparatus 50 according to the present disclosure may be realized by any other appropriate hardware configuration.

### Operation Examples

Hereinafter, operation examples of ultrasound diagnostic system I will be described in detail.

### <Training Phase>

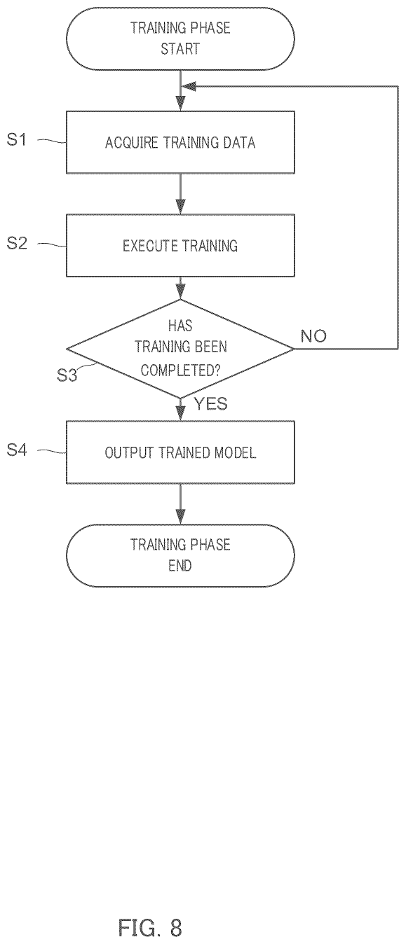

First, an operation example of training apparatus 50 in a training phase will be described. FIG. 8 is a flowchart provided for describing an operation example of training apparatus 50 in the training phase.

In step S1, data acquisition unit 51 of training apparatus 50 acquires training data.

The training data is ultrasound image data obtained from various subjects. In a case where it is assumed that ultrasound diagnostic system 1 is used particularly for a lumbar nerve root block, the training data is desirably ultrasound image data of the lumbar regions of various subjects. Nonetheless, the training data may include ultrasound image data of a region(s) other than the lumbar region.

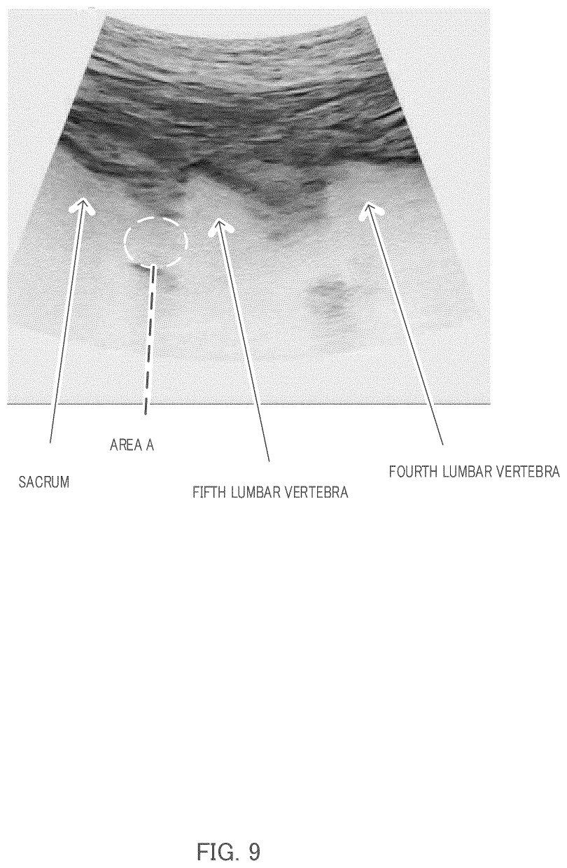

FIG. 9 is a diagram illustrating an exemplary ultrasound image of the lumbar region. FIG. 9 illustrates an exemplary ultrasound image obtained by applying the ultrasound probe to the lumbar region of a subject from the back side of the subject along substantially the median line and emitting ultrasound. The left side of FIG. 9 corresponds to the foot side of the subject, and the right side thereof corresponds to the head side of the subject.

The white area in FIG. 9 is a muscle, fat, or the like of the subject. In addition, the whiter area existing near the interface between the white area and the black area corresponds to bones. FIG. 9 renders no nerve root.

In the example illustrated in FIG. 9, the fourth lumbar vertebra (L4), the fifth lumbar vertebra (L5), and the sacrum are rendered. The L5 nerve root used for a nerve root block exists between the fifth lumbar vertebra and the sacrum (area A illustrated in FIG. 9). As described above, an ultrasound image of training data used in the present disclosure includes a nerve root to be treated and a bone(s) that should be present around the nerve root.

As described above, the training data is constituted by a pair of ultrasound image data on an ultrasound image as exemplified in FIG. 9 and a ground truth label. The ground truth label is data indicating which area in an ultrasound image is a nerve root (in particular, the L5 nerve root). The ground truth label may be assigned in advance to each ultrasound image by the user or a person other than the user.

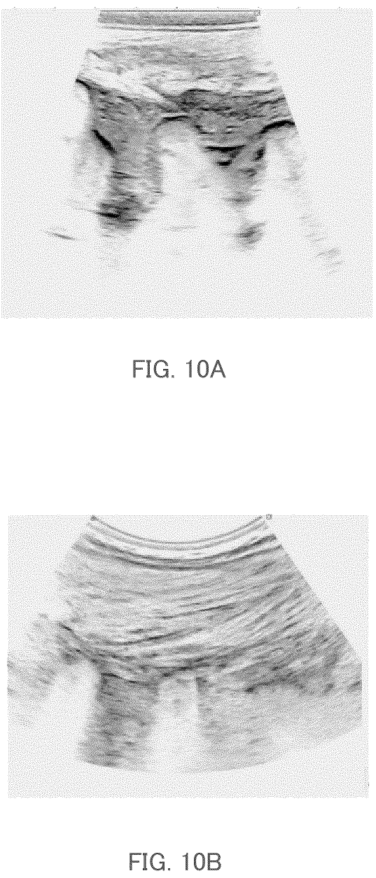

The ultrasound image illustrated in FIG. 9 is an example, and data of any other ultrasound image may be used as the training data according to the present disclosure. FIGS. 10A to 12B are diagrams illustrating other exemplary ultrasound image data as training data.

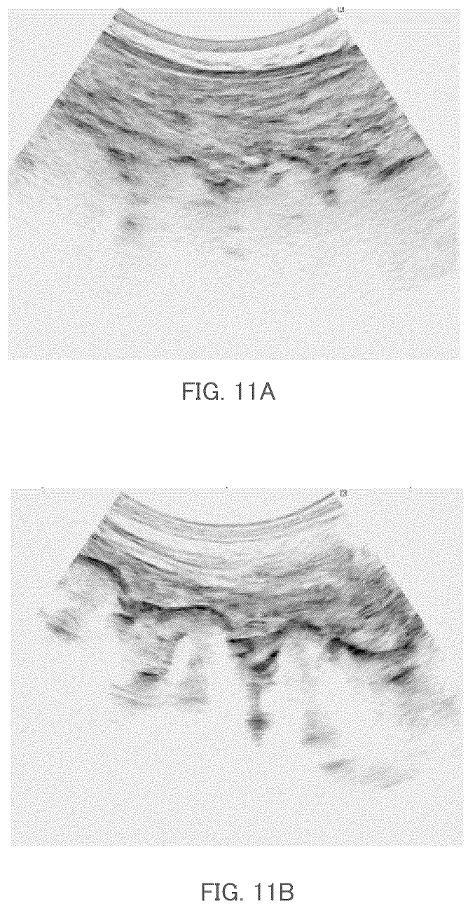

FIGS. 10A to 11B are ultrasound images obtained by applying the ultrasound probe to the median line of a subject from the back side of the subject in the same manner as in the example illustrated in FIG. 9. Even in ultrasound images of similar regions, as illustrated in FIGS. 9, 10A, 10B, 11A and 11B, body tissues appear in the ultrasound images in various manners depending on the subject. Using such a large amount of data of various ultrasound images as training data makes it possible to improve the performance of trained model 10.

FIGS. 11A and 11B illustrate exemplary ultrasound images of the lumbar region, which appear in different manners depending on fat amounts of subjects. FIG. 11A is an exemplary ultrasound image of a subject having relatively much fat, and FIG. 11B is an exemplary ultrasound image of a subject having relatively little fat. Since fat is likely to appear white in an ultrasound image, an image of a subject having relatively much fat is whitish as a whole as compared with an image of a subject having relatively little fat, as illustrated in FIGS. 11A and the 11B.

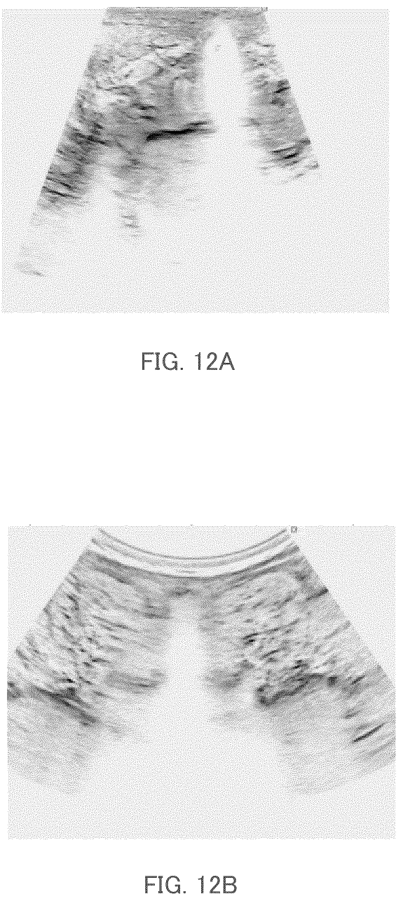

FIGS. 12A and 12B are diagrams illustrating exemplary ultrasound images in which the orientations of cross sections are different. FIGS. 12A and 12B illustrate exemplary ultrasound images in cross sections of the lumbar regions of subjects. These images are obtained in a case where the ultrasound probe is applied to a subject along the right-left direction of the subject from the back side of the subject. In the examples illustrated in FIGS. 12A and 12B, the orientations are different from those in the examples illustrated in FIGS. 9 to 11B, and thus, the body tissues appearing in the images are different. As described above, a pair of an ultrasound image having a different orientation and a training label may be used as training data.

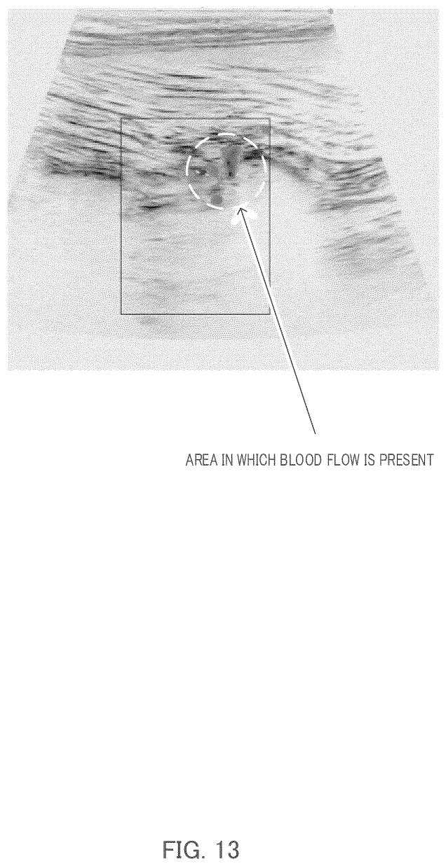

FIG. 13 is a diagram illustrating an exemplary ultrasound image of the lumbar region including blood flow information. The blood flow information is information on a blood flow flowing in a blood vessel (a lumbar artery) running parallel to a nerve root, and indicates a direction in which the blood flow flows with, for example, a color such as red or blue. Thus, in a case where blood flow information is included in an ultrasound image of the lumbar region, it is found that a nerve root is present in the vicinity of a position in which the blood flow information is present, because the nerve root runs parallel to a blood vessel as described above.

In the present disclosure, in addition to the ultrasound images described with reference to FIGS. 9 to 13, data of various ultrasound images may be used as the training data. Furthermore, although the ultrasound images exemplified in FIGS. 9 to 13 are still images, moving images may also be used as training data.

For example, as training data, ultrasound image data of an ultrasound image in which a nerve root is marked by injecting a drug solution or the like for marking the nerve root into a subject at the time of generating the ultrasound image data may be used. According to such an image, it is not necessary for the user to manually assign a ground truth label, and it is possible to significantly reduce the labor for generating training data.

FIG. 8 will be described again. In step S2, training unit 52 of training apparatus 50 trains a machine learning model to be trained, by using the training data acquired in step S1. The training method is not particularly limited in the present disclosure as described above. Since the training is performed using an image in the present disclosure, the backpropagation method or the like suitable for an image may be used as appropriate.

In step S3, training unit 52 determines whether a predetermined completion condition has been completed. The predetermined completion condition may be set in advance by, for example, the user of ultrasound diagnostic system 1. As an example of the completion condition, it is possible to mention completion of learning using a predetermined number of pairs of ultrasound images and ground truth labels.

In a case where training unit 52 determines in step S3 that the training has been completed (step S3: YES), the processing proceeds to step S4. In a case where training unit 52 determines in step S3 that the training has not been completed (step S3: NO), the processing returns to step SI and the training is continued. Note that, an example in which in a case where it is determined that the training has not been completed in step S3, the processing returns to step S1 is illustrated in the flowchart illustrated in FIG. 8, but in the present disclosure, in a case where sufficient training data is acquired in step S1, processing of returning from step S3 to step S2 may be executed.

In step S4, training apparatus 50 outputs trained model 10 (see FIG. 1 or the like), for which the training has been completed, to inference apparatus 200 of ultrasound diagnostic apparatus 100. Thus, the training phase is completed, and an inference phase can be executed.

By performing the training in this way, the machine learning model to be trained becomes trained model 10 capable of outputting output data on a nerve root in response to an input of ultrasound image data. Note that, the output data may be, for example, data itself indicating the position of a nerve root in an ultrasound image, or various data on a nerve root, such as data on the position(s) of a bone(s) existing around a nerve root or data on the position of a blood vessel running parallel to a nerve root.

The output data outputted by trained model 10 may include the above-described plurality of types of data or may include only one type of data.

### <Inference Phase>

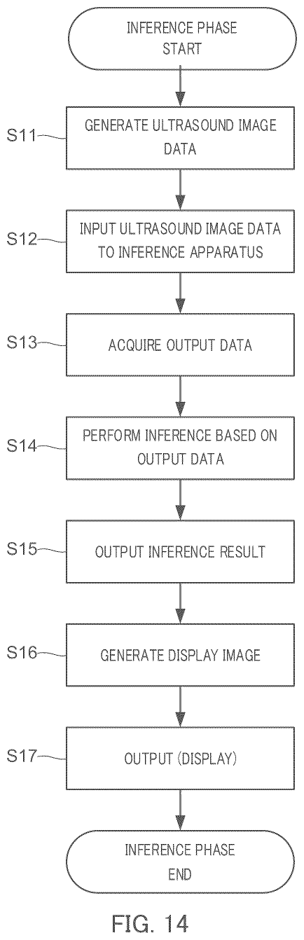

Next, an operation example of ultrasound diagnostic apparatus 100 in the inference phase will be described. FIG. 14 is a flowchart provided for describing an operation example of ultrasound diagnostic apparatus 100 in the inference phase.

The inference phase is executed, for example, in a case where the user performs a nerve root block treatment on a subject by using ultrasound diagnostic apparatus 100.

In step S11, ultrasound image generation unit 1014 (see FIG. 5) of ultrasound diagnostic apparatus 100 generates ultrasound image data based on a reflected echo received from ultrasound probe 1020 pressed against a subject. For example, in a case where a lumbar nerve root block is performed, the user generates an ultrasound image of the lumbar region.

In step S12, ultrasound image generation unit 1014 inputs the generated image data to inference apparatus 200. In a case where the generated ultrasound image is a moving image, ultrasound image generation unit 1014 inputs image data generated for each frame to inference apparatus 200. Thus, image acquisition unit 201 of inference apparatus 200 acquires ultrasound image data to be inferred.

In step S13, inference unit 203 of inference apparatus 200 inputs the ultrasound image data to be inferred to trained model 10 stored in storage unit 204, and acquires output data outputted from trained model 10.

As described above, the output data may include data indicating the position of a nerve root itself as well as a plurality of types of data such as data indicating the position(s) of a bone(s) (such as the fourth lumbar vertebra, the fifth lumbar vertebra, the sacrum, or the like in the case of the lumbar region) that should exist around a nerve root or data indicating the position of a blood vessel (a lumbar artery in the case of the lumbar region) running parallel to a nerve root.

In step S14, inference unit 203 performs inference on a nerve root in an ultrasound image based on the output data.

Specifically, in a case where the output data includes the position of a nerve root to be treated, inference unit 203 specifies the position of the nerve root based on the output data.

Furthermore, in a case where the output data includes the position(s) of a bone(s) that should exist around a nerve root to be treated, inference unit 203 performs inference on the position of the nerve root based on the output data. For example, in the ultrasound image of the lumbar region exemplified in FIG. 9, inference on a position in which a nerve root should be present (area A) is performed based on the position of the fourth lumbar vertebra, the position of the fifth lumbar vertebra, and the position of the sacrum which are indicated by the output data.

Furthermore, in a case where the output data includes the position of a blood vessel that should run parallel to a nerve root to be treated, inference unit 203 performs inference on the position of the nerve root based on the output data. For example, in the example illustrated in FIG. 13, inference unit 203 performs inference on a position, in which a nerve root should be present, around an area in which a blood flow is present.

To improve the accuracy of nerve root inference, it is more desirable that inference unit 203 infer the position of a nerve root in an ultrasound image by combining a plurality of types of inference results using a plurality of types of data included in the output data.

In step S15, inference unit 203 outputs inference result (the position of a nerve root in an ultrasound image) data to display image generation unit 1015.

In step S16, display image generation unit 1015 generates a display image to be displayed on output unit 1016, based on the ultrasound image data acquired from ultrasound image generation unit 1014 and the inference result data indicating the position of a nerve root in an ultrasound image of the ultrasound image data.

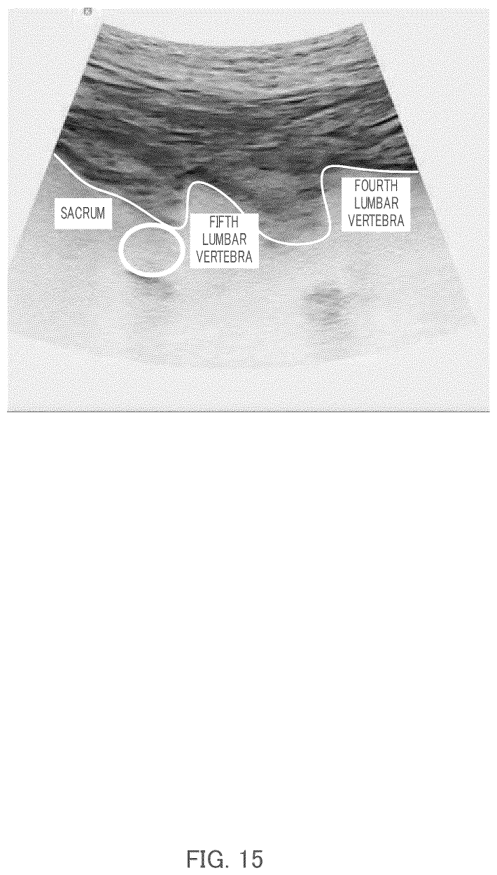

FIG. 15 is a diagram illustrating an exemplary display image. The display image illustrated in FIG. 15 is an image generated by superimposing a display element (mark) indicating the position of a nerve root in the ultrasound image illustrated in FIG. 9. In the example illustrated in FIG. 15, the display element of the white circle indicates the position of a nerve root.

In addition, in the example illustrated in FIG. 15, labels (“fourth lumbar vertebra”, “fifth lumbar vertebra”, and “sacrum”) indicating what are the areas other than the nerve root in the ultrasound image are illustrated. Such labels may not be displayed in the display image in a case where the user does not require the labels.

Note that, the display image illustrated in FIG. 15 is an example, and various display images may be displayed on output unit 1016 in the present disclosure. For example, in order to highlight a display element indicating the position of a nerve root, the display element may be constituted by a frame of a fluorescent color.

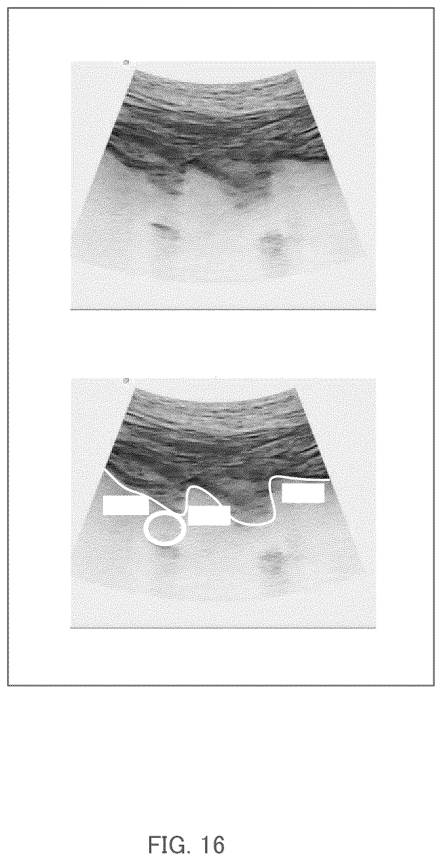

Further, for example, as exemplified in FIG. 16, a display screen may be generated in which an ultrasound image, in which a display element or a label indicating the position of a nerve root is not displayed, and an ultrasound image, in which a display element or a label indicating the position of a nerve root is displayed, are displayed side by side. Although the two images are vertically arranged in FIG. 16, a display screen in which the two images are horizontally arranged may be generated.

In step S17, output unit 1016 outputs (displays) the display image. Thus, the user can accurately grasp the position of a nerve root.

Note that, in a case where an ultrasound image is a moving image, ultrasound diagnostic apparatus 100 repeats step S11 illustrated in FIG. 14 for each frame. Thus, the user can accurately grasp the position of a nerve root in real time.

As described above, inference apparatus 200 performs inference on the position of a nerve root based on output data outputted by a learned model in response to an input of ultrasound image data, and ultrasound diagnostic apparatus 100 generates a display image based on an inference result of inference apparatus 200, thereby making it possible to generate a display image including a display element that accurately indicates the position of a nerve root. Accordingly, the user who performs a nerve root block treatment on a subject can accurately grasp the position of a nerve root even when the user does not elicit a reaction of the subject. Therefore, it is possible to perform a nerve root block with a small burden on the subject.

Hereinabove, the examples of the present disclosure have been described in detail, but the present disclosure is not limited to the above-described specific embodiment, and various modifications and changes can be made within the scope of the gist of the present disclosure described in the claims.

Although embodiments of the present invention have been described and illustrated in detail, the disclosed embodiments are made for purposes of illustration and example only and not limitation. The scope of the present invention should be interpreted by terms of the appended claims.

What is claimed is:

1. An inference apparatus, comprising a hardware processor that:
acquires ultrasound image data on an ultrasound image generated based on a reflected wave of ultrasound transmitted to a subject; and
inputs the ultrasound image data, as data to be inferred, to a trained model and performs inference on a nerve root in the ultrasound image based on output data outputted from the trained model.

2. The inference apparatus according to claim 1, wherein
the hardware processor infers a position of the nerve root based on a position of a bone around the nerve root, the position of the bone being included in the output data outputted from the trained model.

3. The inference apparatus according to claim 2, wherein the hardware processor that:
acquires blood flow information on a blood flow flowing in a blood vessel, the blood flow information being included in the ultrasound image, and
infers the position of the nerve root based on the blood flow information.

4. The inference apparatus according to claim 1, wherein
the trained model is trained so as to output the output data in a case where the ultrasound image data is inputted, by using, as training data, the ultrasound image including a bone existing around the nerve root.

5. The inference apparatus according to claim 4, wherein
the trained model has been trained by using, as the training data, the ultrasound image including at least one bone of a fifth lumbar vertebra, a fourth lumbar vertebra or a sacrum.

6. The inference apparatus according to claim 4, wherein
the trained model has been trained by further using, as the training data, blood flow information on a blood flow flowing in a blood vessel running parallel to the nerve root.

7. The inference apparatus according to claim 1, wherein
the trained model has been trained by using, as training data, the ultrasound image in which the nerve root is marked.

8. An ultrasound diagnostic apparatus, comprising:
the inference apparatus according to claim 1;
an image generator that generates the ultrasound image; and
a display that displays the ultrasound image, and performs displaying on the nerve root based on the inference.

9. The ultrasound diagnostic apparatus according to claim 8, wherein
the display displays, based on the inference, a nerve root position display element indicating a position of the nerve root in a superimposed manner in the ultrasound image.

10. The ultrasound diagnostic apparatus according to claim 9, wherein
the display highlights and displays the nerve root position display element in the ultrasound image.

11. The ultrasound diagnostic apparatus according to claim 9, wherein
the display displays the ultrasound image, on which the nerve root position display element is superimposed, and the ultrasound image, on which the nerve root position display element is not superimposed, side by side.

12. The ultrasound diagnostic apparatus according to claim 9, wherein
the display displays, in an area in which the nerve root position display element is not displayed in the ultrasound image, a label based on the inference.

13. A training apparatus, comprising a hardware processor that:
acquires a pair of ultrasound image data and a ground truth label, the ultrasound image data being data on an ultrasound image, the ground truth label indicating a position of a nerve root in the ultrasound image; and
generates a trained model by using the pair as training data, the trained model being trained so as to output an inference result on the nerve root in response to an input of the ultrasound image data.

14. The training apparatus according to claim 13, wherein
the ultrasound image used as the training data includes a bone existing around the nerve root.

15. The training apparatus according to claim 14, wherein
the ultrasound image used as the training data includes at least one bone of a fifth lumbar vertebra, a fourth lumbar vertebra or a sacrum, each of which exists around the nerve root.

16. The training apparatus according to claim 14, wherein
the ultrasound image used as the training data further includes blood flow information on a blood flow flowing through a blood vessel running parallel to the nerve root.

17. An inference method executed by a computer that forms an inference apparatus, the inference method comprising:
acquiring ultrasound image data on an ultrasound image generated based on a reflected wave of ultrasound transmitted to a subject; and
inputting the ultrasound image data, as data to be inferred, to a trained model, and performing inference on a nerve root in the ultrasound image based on an inference result outputted from the trained model.

18. A display method executed by a computer that forms an ultrasound diagnostic apparatus, the display method comprising:
transmitting ultrasound to a subject, and generating an ultrasound image based on a reflected wave of the ultrasound;
acquiring ultrasound image data on the ultrasound image having been generated;
inputting the ultrasound image data, as data to be inferred, to a trained model, and performing inference on a nerve root in the ultrasound image based on output data outputted from the trained model; and
displaying the ultrasound image, and performing displaying on the nerve root based on the inference.

19. A non transitory recording medium storing a computer readable program causing a computer that forms an inference apparatus, the program causing the computer to perform:
acquiring ultrasound image data on an ultrasound image generated based on a reflected wave of ultrasound transmitted to a subject; and
inputting the ultrasound image data, as data to be inferred, to a trained model, and performing inference on a nerve root in the ultrasound image based on output data outputted from the trained model.

20. A non transitory recording medium storing a computer readable program causing a computer that forms an ultrasound diagnostic apparatus, the program causing the computer to perform:
transmitting ultrasound to a subject and generating an ultrasound image based on a reflected wave of the ultrasound;
acquiring ultrasound image data on the ultrasound image having been generated;
inputting the ultrasound image data, as data to be inferred, to a trained model, and performing inference on a nerve root in the ultrasound image based on output data outputted from the trained model; and
displaying the ultrasound image, and performing displaying on the nerve root based on the inference.
